(19) United States
(12) Patent Application Publication
(10) Pub. No.: US 2023/0309823 A1
(43) Pub. Date: Oct. 5, 2023

(54) DIAGNOSTIC CLASSIFICATION OF CORNEAL DISEASES BASED ON ARTIFICIAL INTELLIGENCE

(71) Applicant: Oregon Health & Science University, Portland, OR (US)

(72) Inventors: Yan Li, Portland, OR (US); Elias Pavlatos, Portland, OR (US); Xubo Song, Portland, OR (US); David Huang, Portland, OR (US)

(73) Assignee: Oregon Health & Science University, Portland, OR (US)

(21) Appl. No.: 18/129,767

(22) Filed: Mar. 31, 2023

Related U.S. Application Data
(60) Provisional application No. 63/326,739, filed on Apr. 1, 2022.

(51) Int. Cl. A61B 3/107; G06T 7/60

(52) U.S. Cl. CPC A61B 3/107; G06T 7/60; G06T 2207/10101; G06T 2207/30041

(57) ABSTRACT

Disclosed are artificial intelligence (AI) based systems and methods for characterizing corneal shape abnormalities. The methods and systems of the present disclosure utilize AI models comprising neural networks for disease classification based on maps of corneal shape, thickness, and reflectance. These methods may be used to differentiate corneas having keratoconus from other conditions which may cause distortion of corneal shape, such as warpage of the cornea. The present system is amenable to automation and may be implemented in an integrated system or provided in the form of software encoded on a computer-readable medium.

### CROSS-REFERENCE TO RELATED APPLICATION

The present application claims priority to U.S. Provisional Patent Application No. 63/326,739, filed Apr. 1, 2022, entitled “DIAGNOSTIC CLASSIFICATION OF CORNEAL DISEASES BASED ON ARTIFICIAL INTELLIGENCE”, the entire disclosure of which is hereby incorporated by reference.

### ACKNOWLEDGEMENT OF GOVERNMENT SUPPORT

This invention was made with government support under R01 EY028755 and R01 EY029023 awarded by the National Institutes of Health. The government has certain rights in the invention.

### TECHNICAL FIELD

The present disclosure generally relates to the field of ophthalmology. In particular, artificial intelligence based systems and methods for the characterization and classification of corneal shape abnormalities are disclosed.

### BACKGROUND

Conventional corneal topography is an important tool in the recognition of forme fruste (pre-clinical) keratoconus (FFK), an important risk factor for post-LASIK ectasia, a serious complication of corneal refractive surgery. However, the recognition of FFK on topographic displays such as axial power and tangential power maps is a complex exercise because FFK can manifest as many possible patterns of distortion. Several tools have been developed to make the detection of FFK using corneal topographic data more reliable. The mean curvature (also referred to as mean power) map, for example, has been shown to better characterize keratoconus than the conventional axial and tangential power maps. This is because the mean curvature map contains information about both the radial and azimuthal curvature changes that occur in keratoconus, but is not confounded by regular astigmatism. In addition, more recent studies have shown that corneal pachymetry (i.e., corneal thickness) and epithelial thickness maps can be more sensitive than topography for keratoconus diagnosis.

None of these corneal maps on their own, however, can differentiate keratoconus from other corneal pathologies with similar topographic patterns, such as contact lens-related warpage, dry eye disease, and Fuchs' endothelial dystrophy. Contact lens-related warpage of the cornea is of particular significance due to the prevalence of contact lens use in the population. Because many LASIK candidates are contact lens wearers, the distinction between contact lens-related warpage and FFK is a common diagnostic challenge faced by clinicians to ensure that post-LASIK ectasia outcomes are avoided. Fuchs' dystrophy is a degenerative disease of the corneal endothelium with accumulation of guttae (focal deposits) between the corneal endothelium and Descemet's membrane, endothelial cell loss, followed by corneal edema and vision loss. It is important to identify this progressive disease early, as the results of surgical intervention is better in its earlier stages. An undiagnosed early Fuchs' dystrophy imposes challenge on eye banking, as it carries a risk of transplanting patients with diseased corneal grafts. Therefore, there still exists a need for reliable systems and methods to accurately diagnose and/or differentiate between corneal conditions such as FFK, contact lens-related warpage, dry eye disease, and Fuchs' endothelial dystrophy.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIGS. 1A and 1B show topography maps from optical coherence tomography (OCT) and Scheimpflug tomography (Pentacam), respectively, for a cornea with keratoconus in accordance with an embodiment of the present disclosure. The Pentacam maps in FIG. 1B are cropped to be the same size as the OCT maps in FIG. 1A. The color scale used by the Pentacam is replicated for the OCT maps.

FIG. 2 illustrates a cross-sectional corneal optical coherence tomography (OCT) image (average of 5 repeated frames) showing an epithelial surface, an endothelium, and a Bowman's layer in accordance with various embodiments.

FIG. 3 illustrates maps of pachymetry, epithelial thickness, posterior float elevation, and posterior mean curvature for a cornea with keratoconus in accordance with an embodiment of the present disclosure.

FIG. 4 shows maps of enhanced posterior float elevation for a cornea with Fuchs' endothelial dystrophy, in accordance with an embodiment of the present disclosure.

FIG. 5 illustrates an OCT image and an endothelial slab reflectance intensity plot for each of a normal cornea and a cornea having Fuchs' dystrophy.

FIG. 6 shows schematic diagrams of an example system comprising an artificial intelligence (AI) model configured to perform corneal classification in accordance with the present disclosure.

FIG. 7 illustrates downsampling of an example pachymetry map obtained from an OCT of a cornea for inputting into the AI model of the example system of FIG. 6.

FIG. 8 illustrates characteristic downsampled maps of pachymetry, epithelial thickness, posterior mean curvature, enhanced posterior float elevation, and reflectance of the Descemet's layer and endothelium for respective corneas having normal, Keratoconus, or Fuchs' endothelial dystrophy, in accordance with various embodiments. The characteristic downsampled OCT maps may be used for inputting into the AI model of the example system of FIG. 6.

FIG. 9 is a schematic diagram of an example system including an AI model having specialized convolutional/pooling layers that connect to fully connected layers according to various embodiments.

FIG. 10 is a table that shows results of a 5-fold cross-validation in accordance with various embodiments of the present disclosure.

FIGS. 11A-11C illustrate an OCT scan pattern to map epithelial thickness, pachymetry and corneal topographies, in accordance with Example 1 described herein.

FIG. 11A illustrates a radial scan pattern (diameter 6 mm, 8 meridians, 1,020 axial scans each meridional radial line, repeated 5 times, acquisition time 0.6 second). FIG. 11B illustrates cross-sectional OCT (average of 5 repeated frames). FIG. 11C illustrates maps of pachymetry, epithelial thickness, anterior mean curvature and posterior mean curvature for a manifest keratoconus eye.

FIG. 12 illustrates average class activation maps for each group of subjects (normal, warpage, manifest keratoconus, subclinical keratoconus, and forme fruste keratoconus) in the study conducted in accordance with Example 1 described herein.

FIG. 13 schematically shows an example system for processing OCT datasets in accordance with the disclosure.

FIG. 14 schematically shows an example of a computing system in accordance with the disclosure.

### DETAILED DESCRIPTION OF DISCLOSED EMBODIMENTS

Disclosed herein are artificial intelligence (AI) based systems and methods for characterizing corneal shape abnormalities. In some embodiments, the methods may include combining the features represented in several types of corneal maps and utilizing them as inputs for machine learning models to detect and classify corneal irregularities. In some embodiments, metrics derived from the corneal maps or the maps themselves may be combined and used as inputs for AI-based models to classify a subject's cornea. The corneal maps may be generated from data derived from one or more imaging modalities, including optical coherence tomography (OCT), Scheimpflug camera-based corneal tomography, Placido topography, slit-scanning tomography, ultrasound imaging, or any other suitable means known in the art for measuring corneal properties. Specific corneal maps may include, but are not limited to, corneal topography maps (elevation, axial power, tangential power, or mean curvature), corneal thickness maps (pachymetry, epithelial thickness, or stromal thickness), and corneal reflectance maps (epithelial reflectance, corneal stromal reflectance, or Descemet's membrane and endothelium reflectance).

An OCT image processing algorithm may be used to segment OCT images of the cornea and generate maps of shape (e.g., corneal anterior, posterior, and subepithelial surfaces), thickness (e.g., overall cornea, epithelial, stromal, Descemet's, and endothelial sublayers), and reflectance (e.g., epithelial & Bowman's layer, anteerior stromal, mid-stromal, posterior stromal, and endothelium & Descemet's membrane). Plots of reflectance variation may also be generated at various depths through the corneal thickness. In embodiments, AI models comprising one or more neural networks (e.g., convolutional neural network) may be used for disease classification based on OCT maps of corneal shape, thickness, and reflectance. Additionally, AI models may detect depth-dependent irregularities in reflectance intensity in the OCT images of the cornea. Diagnostic metrics may be formulated by modeling characteristic features and combining information from at least two different types of measurements. The maps and/or metrics may be used to train AI models to detect and classify irregular corneas.

An example method for classifying shape abnormalities of the cornea using the disclosed subject matter generally comprises: generating corneal maps of a subject's cornea; providing the corneal maps and/or metrics derived from the corneal maps of the subject's cornea as inputs (e.g., via an interface) to an artificial intelligence model comprising one or more neural networks; obtaining, via the one or more neural networks, one or more outputs related to presence of at least one condition, the one or more neural networks generating the one or more outputs based on the corneal maps and/or metrics derived from the corneal maps; and classifying the subject's cornea based on the one or more outputs generated by the one or more neural networks of the AI model.

In embodiments, the disclosed methods may provide an automated system for diagnosing conditions in the cornea and for differentiating pathologic and non-pathologic conditions. Further embodiments also include a computer-readable medium encoding the disclosed methods.

The systems and methods of the present disclosure may allow enhanced diagnostic capability and reduce computational demand. The present systems and methods may accurately identify abnormal tissue within the cornea and diagnose diseases causing irregular corneal structure. Furthermore, the disclosed systems and methods may be incorporated into commercial anterior eye OCT systems to aid in diagnostic decision making.

In the following detailed description, reference is made to the accompanying drawings which form a part hereof, and in which are shown by way of illustration embodiments that can be practiced. It is to be understood that other embodiments can be utilized and structural or logical changes can be made without departing from the scope. Therefore, the following detailed description is not to be taken in a limiting sense, and the scope of embodiments is defined by the appended claims and their equivalents.

Various operations can be described as multiple discrete operations in turn, in a manner that can be helpful in understanding embodiments; however, the order of description should not be construed to imply that these operations are order dependent.

The description may use the terms “embodiment” or “embodiments,” which may each refer to one or more of the same or different embodiments. Furthermore, the terms “comprising,” “including,” “having,” and the like, as used with respect to embodiments, are synonymous.

Unless otherwise noted or explained, all technical and scientific terms used herein are used according to conventional usage and have the same meaning as commonly understood by one of ordinary skill in the art which the disclosure belongs. Although methods, systems, and apparatuses/materials similar or equivalent to those described herein can be used in the practice or testing of the present disclosure, suitable methods, systems, and apparatuses/materials are described below.

All publications, patent applications, patents, and other references mentioned herein are incorporated by reference in their entirety. In case of conflict, the present specification, including explanation of terms, will control. In addition, the methods, systems, apparatuses, materials, and examples are illustrative only and not intended to be limiting.

Corneal topography is an important technology for measuring the shape of the cornea. Early technologies used a Placido disc approach which involves constructing the shape of the anterior cornea by measuring how concentric rings of light reflect off of its surface (Busin M, Wilmanns I, and Spitznas M. Automated corneal topography: Computerized analysis of photokeratoscope images. Graefe's Arch Clin Exp Ophthalmol 1989; 227(3):230-236, incorporated by reference herein). Measurement of both anterior and posterior corneal topography was made possible by the application of the scanning slit technique (e.g. Orbscan, Bausch & Lomb, Bridgewater, New Jersey) or the Scheimpflug imaging principle (Oliveira C M, Ribeiro C, and Franco S. Corneal imaging with slit-scanning and Scheimpflug imaging techniques. Aust J Optom 2011; 94(1):33-42, incorporated by reference herein). More recently, topographs based on OCT have been developed. OCT, because of its high resolution, not only better detects the faint boundary of the posterior cornea, but also measures epithelial thickness (Li Y, et al. Corneal epithelial thickness mapping by Fourier-domain optical coherence tomography in normal and keratoconic eyes. Ophthalmology 2012; 119(12): 2425-2433, incorporated by reference herein). Additionally, OCT scanning speed allows it to complete tomographic scans faster than the nearly 2 seconds required by the Scheimpflug camera-based Pentacam system.

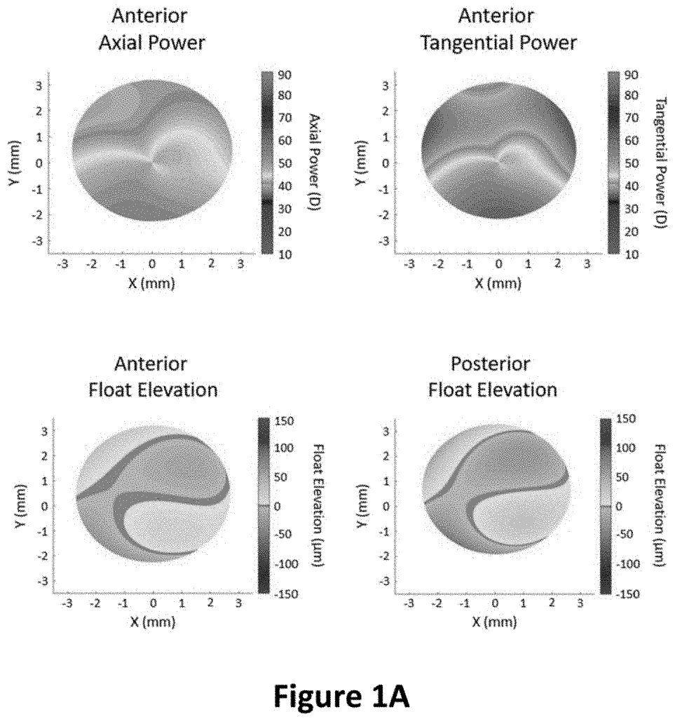

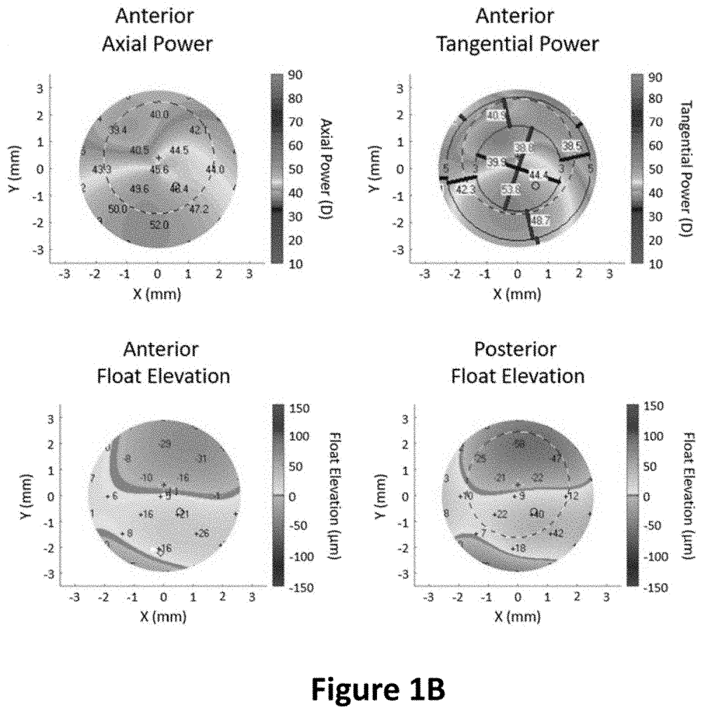

Topography maps generated from the scans of a cornea with keratoconus are displayed in FIGS. 1A and 1B. Compared to normal corneas, the keratoconic cornea has a higher surface curvature which is illustrated by the corneal power maps. The axial and tangential power maps display good agreement between OCT and Pentacam. The float elevation map values differ between OCT and Pentacam because of the different fitting zone diameters that are used to calculate the best-fit sphere. The region of high curvature in the inferior region of the cornea correspond roughly with the area of large positive float elevation (Pavlatos E, Huang D, and Li Y Eye motion correction algorithm for OCT-based corneal topography. Biomedical Optics Express 2020; 11(12): 7343-7356, incorporated by reference herein).

Corneal topography is an essential part of LASIK pre-operative workup to detect FFK and keratoconus, the most important risk factors of post-LASIK ectasia. However, topography is not sensitive to the very early stages of keratoconus when topographic steepening is masked by focal epithelial thinning. Furthermore, contact lens-related warpage can sometimes manifest as inferior steepening on topography with a pattern that is indistinguishable from keratoconus or FFK.

As an alternative to topography-based measures, diagnostic parameters based on OCT corneal pachymetry and epithelial thickness maps have been developed to help detect early keratoconus. These studies have shown that pattern standard deviation (PSD) based on OCT epithelial thickness—Epithelial PSD—is a particularly effective parameter for differentiating keratoconus from normal eyes, including manifestations of subclinical keratoconus. For example, in a group of 50 subclinical keratoconus (CDVA 20/20 or better) and 150 normal control eyes, Epithelial PSD was able to detect early keratoconus with sensitivity of 96% at 100% specificity (Li Y, Chamberlain W, Tan O, et al. Subclinical keratoconus detection by pattern analysis of corneal and epithelial thickness maps with optical coherence tomography. J Cataract Refract Surg 2016; 42(2):284-95, incorporated by reference herein). Furthermore, Epithelial PSD has been shown to be effective in detecting abnormality in keratometry, I-S (inferior-superior dioptric asymmetry), skew percentage, astigmatism (KISA)-normal FFK eyes.

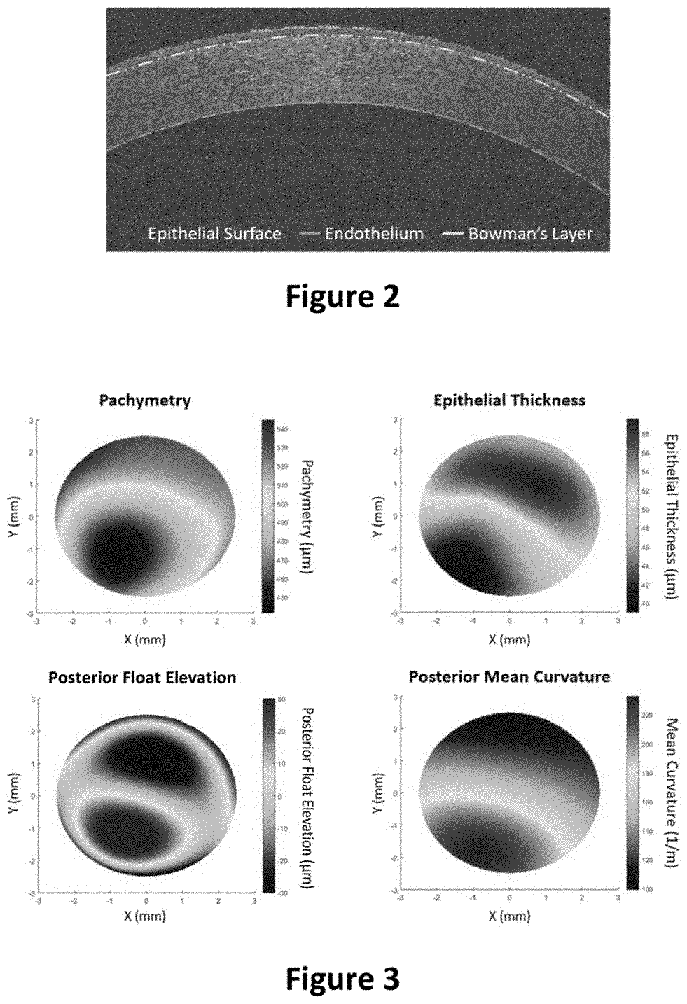

Shown in FIG. 2 is a cross-sectional corneal OCT image (average of 5 repeated frames) indicating an epithelial surface, an endothelium, and a Bowman's layer in accordance with various embodiments. FIG. 3 illustrates a set of example parameter maps (e.g., pachymetry, epithelial thickness, posterior float elevation, and posterior mean curvature) for a cornea with keratoconus. As shown, the OCT epithelial map indicates focal thinning in keratoconus. The pachymetry map also shows focal thinning in case of keratoconus. FFK is often diagnosed by inferior focal steepening on anterior axial topography. A posterior steepening (in posterior mean curvature map) is only found in FFK. Thus, early FFK may be recognized by focal anterior/posterior steepening and epithelial/pachymetric thinning.

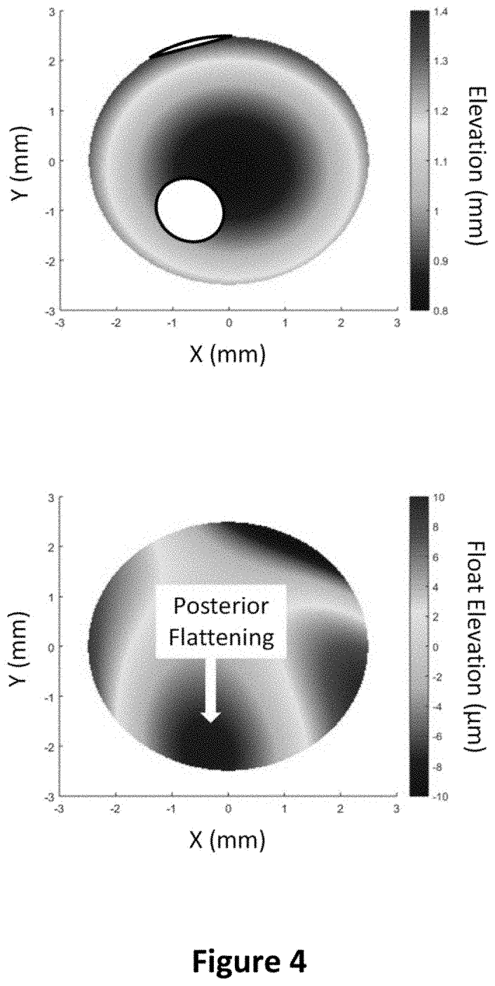

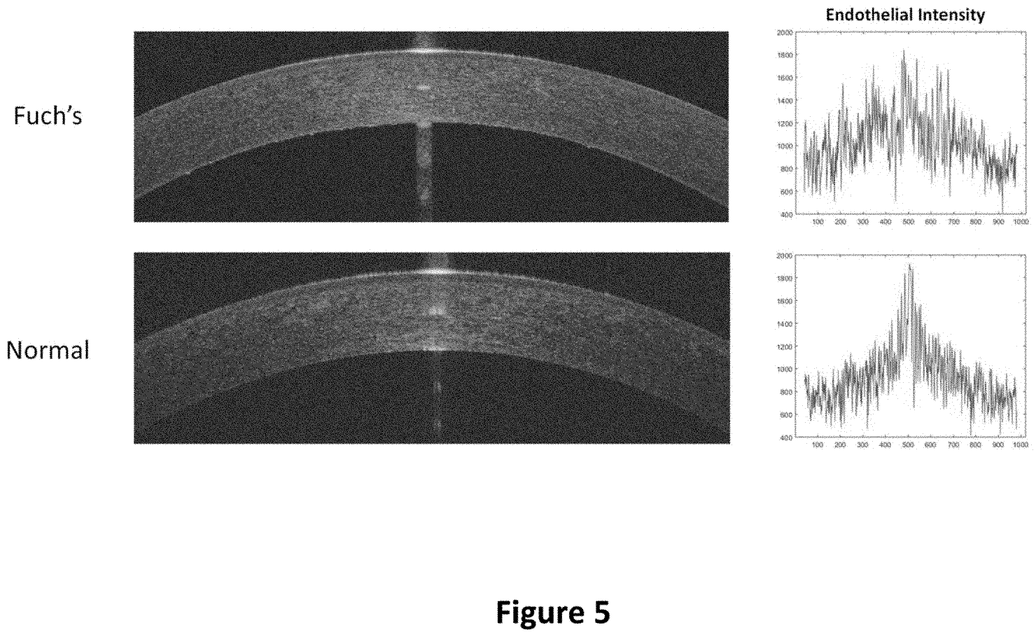

FIG. 4 illustrates maps of enhanced posterior float elevation for a cornea with Fuchs' endothelial dystrophy, in accordance with an embodiment of the present disclosure. An elevation in the thickest and thinnest 5% areas of the elevation map may be excluded. As shown, the enhanced posterior float elevation map indicates posterior flattening in case of Fuchs' endothelial dystrophy. Additionally, FIG. 5 illustrates OCT images and endothelial slab intensity plots differentiating between a normal cornea and a cornea with Fuchs' dystrophy. The OCT images of the cornea may be used to measure spatial variations in reflectance intensity. While the illustrated example uses reflectance intensity, other examples may use signal intensity, image brightness, and so forth.

While Epithelial PSD is very sensitive at detecting the focal epithelial thinning that masks early ectasia on anterior topography, it is also very sensitive at detecting the uneven epithelium variation that characterizes contact lens-related warpage and other corneal surface distortions. Thus, Epithelial PSD alone is unable to effectively differentiate FFK from contact lens-related warpage or other corneal abnormalities (such as Fuchs' dystrophy). Consequently, alternate approaches are needed for effective diagnosis. One approach is to combine information from multiple different corneal maps to characterize corneal shape abnormalities using an artificial intelligence (AI) model.

AI entails the development of computer systems capable of performing tasks that requires human intelligence, such as visual perception, speech recognition, and decision-making. These tasks need cognitive functions associated with human minds, namely learning and problem solving. Machine learning is a subset of AI. Machine learning may be implemented utilizing Deep Learning (DL). DL is a machine learning method that employs mathematical models called neural networks. Neural networks may include large number of layers that attempt to mimic the human brain. In operation, DL attempts to extract complex hierarchal features and patterns present in large datasets. These features may then be merged together using neural networks to represent the model of the data.

Described herein are AI-based systems and methods for corneal diagnosis, such as assisting in the diagnosis or treatment of corneal disease or conditions. The AI-based systems and methods utilize machine learning. For example, systems and methods may utilize AI models in machine learning to automate diagnosis, for example utilizing deep learning models. The systems and methods described herein may be implemented as standalone or integrated applications for processing images or maps such as OCT images, thickness maps, curvature maps, topography maps, tomography maps, elevation maps, or reflectance maps of the human cornea and/or the anterior segment of the eye using Artificial Intelligence models associated with image data. One method may include obtaining a model that takes diagnostic metrics derived from the OCT maps of corneal shape, thickness, and reflectance intensity as input and outputs the diagnosis and/or classification of corneal irregularities. The diagnosis may include the prediction of the disease or condition and the detection of the important features in the input maps. Another method may include obtaining a model that combines OCT corneal topography and thickness maps as input and outputs the diagnosis of the cornea. The diagnosis may include the prediction of the disease or condition and the detection of the important regions in the input OCT maps.

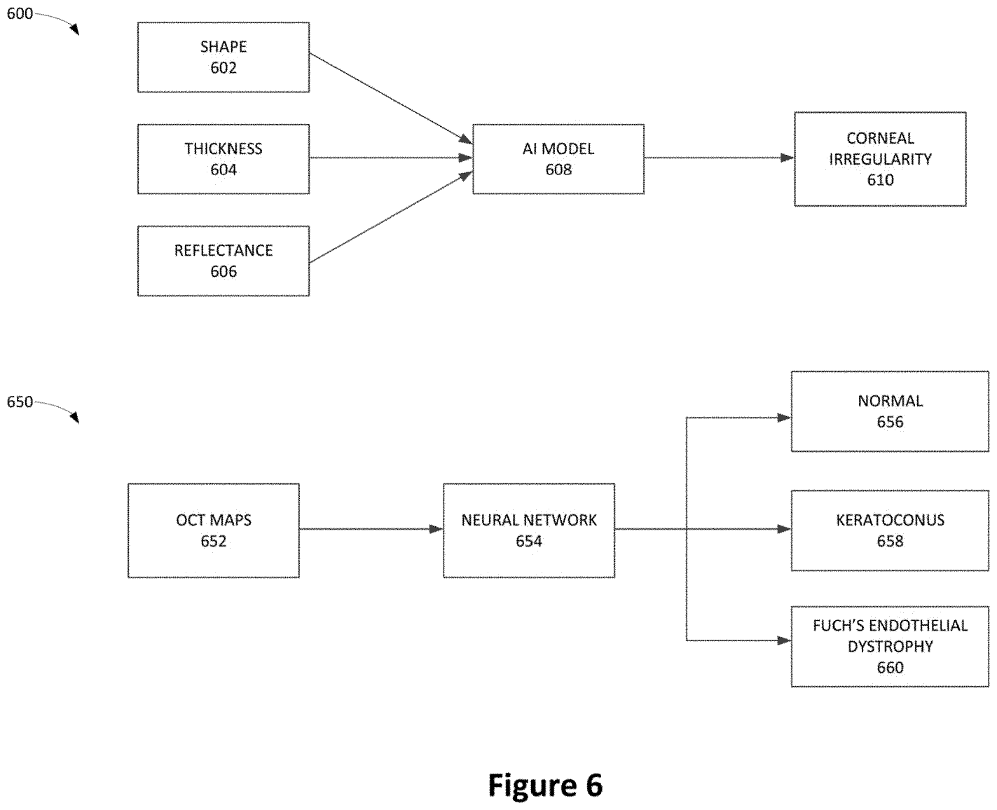

FIG. 6 schematically illustrates embodiments of a system 600 and a system 650, each comprising an AI model configured to perform corneal classification in accordance with the present disclosure. Broadly, each of the system 600 and the system 650 receives data input which is processed through one or more AI models to generate an output prediction. The system 600 and 650 may receive the data input via an interface with one or more processors (e.g., of a local computer) that obtains and/or generates the one or more of the data inputs (e.g., maps and/or metrics). In some embodiments, some or all of the AI model may be hosted in the local computer. Additionally, or alternatively, some or all of the AI model may be implemented in the cloud and/or another remote computing resource.

In operation, the system 600 and the system 650 may execute the prediction operation to diagnose the presence of a certain pathology or its absence in the human cornea or anterior segment of the eye. The data input may include various data such as high resolution images of the cornea, corneal maps, and/or metrics derived from the corneal maps. Data inputs may also include data inputs used for training, testing, and/or tuning the one or more AI models for generating predictions or other system outputs (e.g., detect and classify corneal irregularities). In some embodiments, the system 600 and the system 650 are configured to predict one or more diseases or conditions, if any, in the human cornea and/or predict the severity of disease or condition using an input data. Herein, the terms condition and disease, with respect to the cornea, may be used interchangeably.

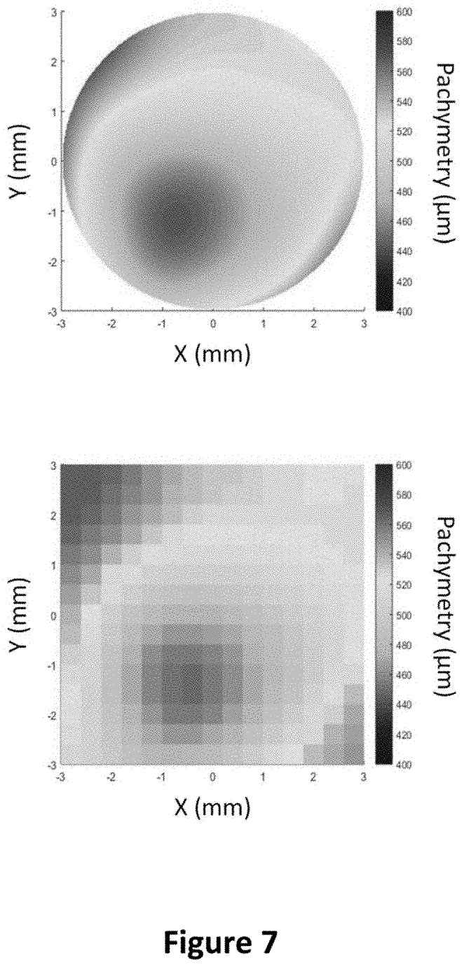

As introduced above, the input data may include images or maps. In various embodiments, and with further reference to FIGS. 7-10, the system 600 and the system 650 may employ AI techniques in OCT. In one example, the system 600 may include an AI model 608. The AI model 608, or one or more sub-models thereof, may take an input such as one or more of the OCT maps of corneal shape 602, corneal thickness 604, and/or reflectance 606. The maps may be generated from images of the cornea using an OCT image processing algorithm. The maps of corneal shape 602 may further include the shapes of corneal anterior, posterior, and sub-epithelial surfaces. The maps of corneal thickness 604 may further include the thickness of overall cornea, epithelial, stromal, Descemet's, and endothelial sublayers. The maps of reflectance 606 may further include the reflectance variation of epithelial/Bowman's, stromal, and endothelial/Descemet's layers. Plots of reflectance variation may also be generated at various depths through the corneal thickness, in some embodiments. An output of a prediction about corneal irregularity 610 may be generated by the AI model 608 based on the input maps. For example, the system 600 may generate a diagnosis for keratoconus.

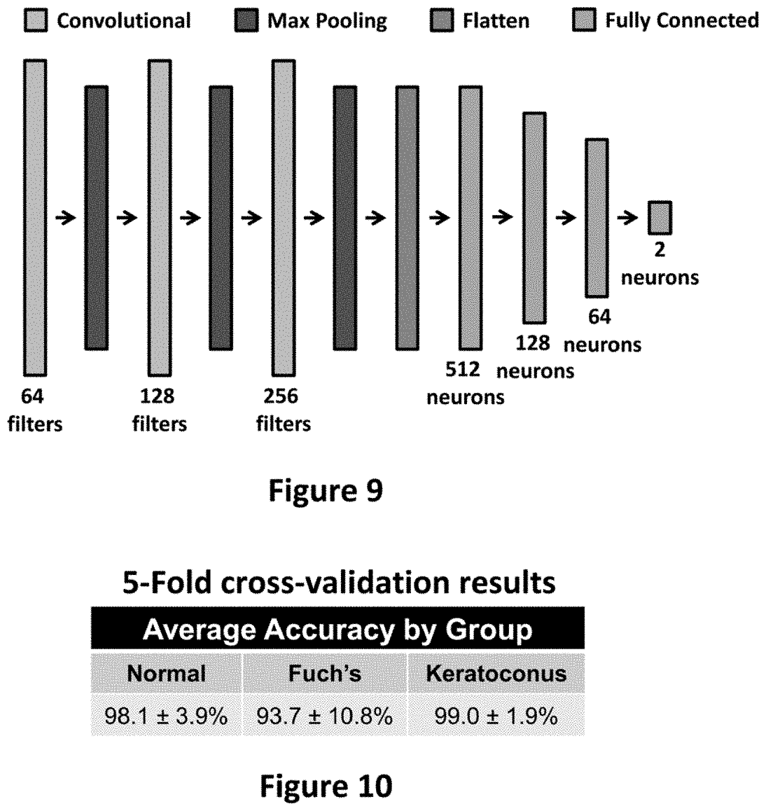

In another example, the system 650 may include an AI model comprising a neural network 654. The neural network 654 may be a convolutional neural network (CNN), which will be described in more details in FIG. 9. As described previously, input data may include OCT maps 652 such as curvature maps, topography maps, tomography maps, or elevation maps (e.g., as shown in FIGS. 1A-4). In some examples, input data may include a map of the cornea representing contours or curvature of the cornea or one or more of its layers. In a further example, the input data may include a map representing relative contour, curvature or elevation of the cornea or one or more of its layers relative to each other or relative to a defined sphere, surface, or curve. In some examples, the input data may include an elevation map of an anterior, posterior, or sub-epithelial surface of the cornea. In some examples, the input data may include an axial power map of an anterior, posterior, or sub-epithelial surface of the cornea. In some examples, the input data may include a tangential power map of an anterior, posterior, or sub-epithelial surface of the cornea. In some examples, the input data may include a mean curvature map of an anterior, posterior, or sub-epithelial surface of the cornea. In yet other examples, input data may include the metrics derived from the OCT maps for AI models comprising the neural network 654. These maps may be produced by application of mapping techniques to images such as OCT images or other high-definition images. Some maps may be produced by devices such as corneal Placido disc topographers or Pentacam® rotating Scheimpflug cameras. Diagnostic metrics may be formulated by modeling characteristic features and combining information from at least two different types of measurements. The maps and/or metrics are used to train AI models to detect and classify irregular corneas in accordance with the embodiments of the present disclosure. For example, as indicated by the system 650, the neural network 654 may classify cornea and generate any one of the three outputs based on the input OCT maps 652. The output may be normal cornea 656, cornea with keratoconus 658, or cornea with Fuchs' endothelial dystrophy 660, in one example. In some examples, the system 650 may detect and/or classify other corneal diseases and generate output accordingly. As discussed herein, Keratoconus is one example of a corneal condition where a specific pattern of thickening of the epithelium at the area where the stroma and Bowman's layers may be thinned. These patterns of relative thickening and thinning may be difficult to represent with an index but may be detected by the AI model that looks at the thickness data of all the layers at the same time and detects such patterns.

In some embodiments, the AI model described herein may include a supervised or unsupervised machine learning process, which may include supervised and unsupervised portions. For example, the AI model (e.g., as shown in FIG. 6) may include one or more AI models configured for supervised learning wherein data examples along with associated labels and annotations are used to train the system. In another or further example, the AI model may include one or more AI models configured for unsupervised learning wherein data examples without any labels or annotations are used to train the system.

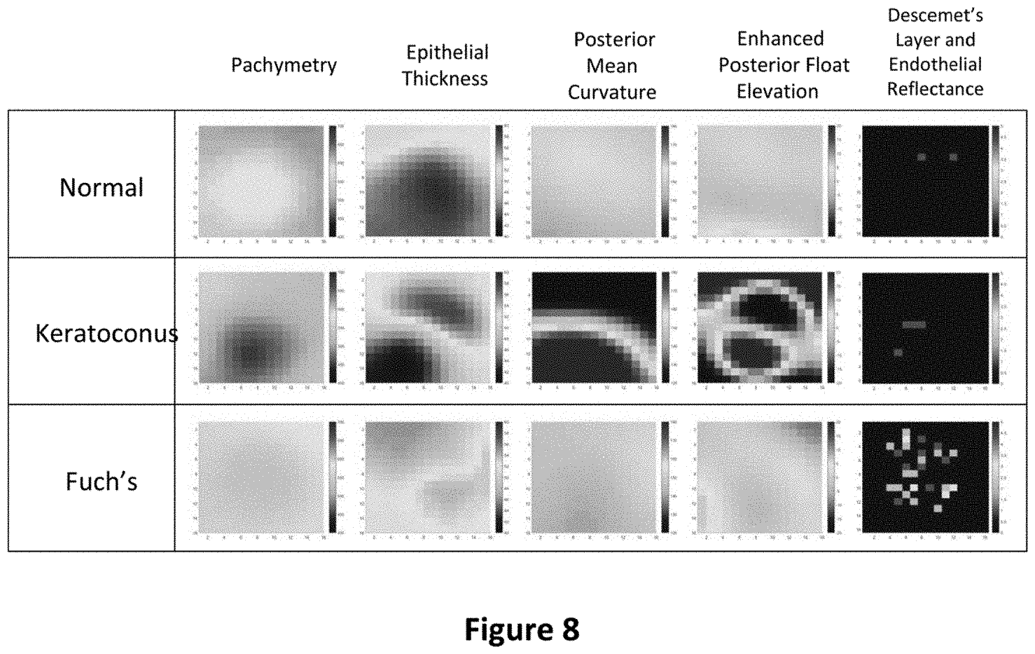

FIG. 7 illustrates downsampling of an example pachymetry map obtained from an OCT of a cornea for inputting into the AI model of the system of FIG. 6. The top image shows the example pachymetry map before downsampling, while the bottom image indicates the downsampled pachymetry map in FIG. 7. The downsampled pachymetry map may be used as an input for the AI model including a supervised machine learning algorithm, such as K-Nearest Neighbour, for classification. FIG. 8 illustrates characteristic downsampled maps of each of pachymetry, epithelial thickness, posterior mean curvature, enhanced posterior float elevation, and accumulation of guttae for respective corneas having normal, Keratoconus, or Fuchs' endothelial dystrophy, in accordance with various embodiments. The characteristic downsampled OCT maps shown in FIG. 8 may be used as input data for the AI model (comprising neural network) of the example system of FIG. 6.

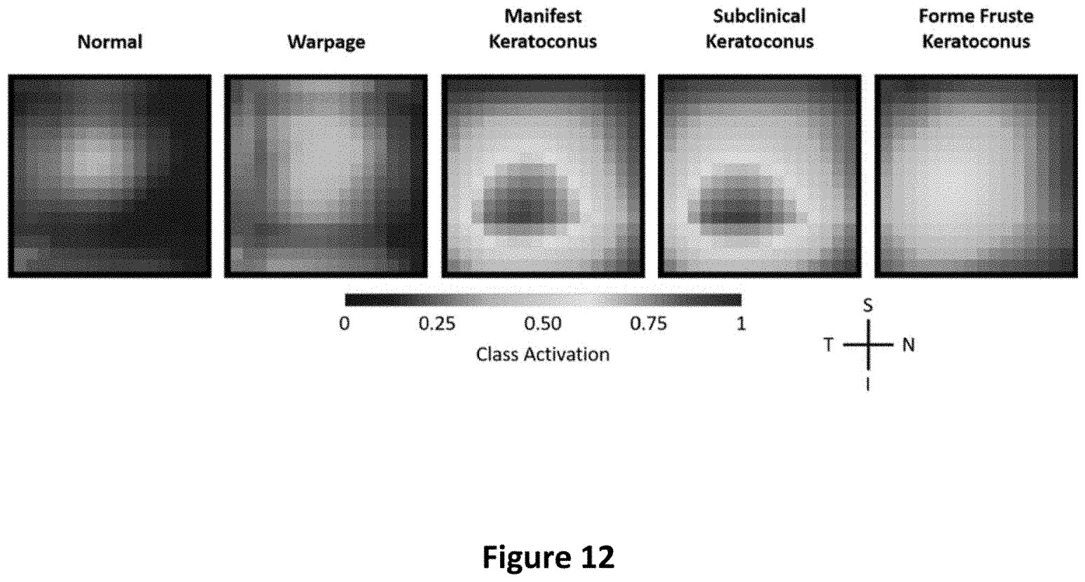

A class activation map for a particular category may indicate the discriminative image regions used by the neural network, e.g., CNN to identify that category. Class activation maps may not only highlight the importance of the image region to the prediction, but may also be used to interpret the prediction decision made by the CNN. FIG. 12 illustrates example class activation maps in accordance with some embodiments.

In various embodiments, a system may include an AI model comprising a convolutional neural network (CNN). As depicted in FIG. 9, a schematic diagram of CNN comprises specialized convolutional/pooling layers that connect to fully connected layers, according to various embodiments. The convolutional neural network may generally include an input layer, a plurality of hidden layers, and an output layer. The plurality of hidden layers may include one or more convolutional layers, one or more pooling layers, one or more fully connected layers, an output layer comprising one or more classification layers, or combinations thereof.

The CNN may take advantage of the dimensional structure of input data by connecting nodes only to a small region of the input data, which may be selected using the dimensionality of the input data, for example. These regions may be referred to as local receptive fields. Sets of nodes may be referred to as a feature map wherein each node may be connected to a receptive field of nodes below. The feature maps may have shared weights such that each node of a given feature map (which corresponds to the same feature, but shifted across the data dimensions) is constrained to have the same input weights and biases as the other nodes of that feature map. A given layer may have multiple feature maps, each able to correspond to a different feature. Layers containing multiple feature maps are referred to as convolutional layers because the output of a node in the layer may include a convolution operation performed on the inputs.

A pooling layer may function to reduce the size of the output of a set of nodes, which is typically the output of a convolutional layer, but pooling layers may be used for any set of nodes, e.g., on top of input nodes. For example, a pooling layer may take the maximum or average activation of a set of nodes as an output, referred to as max-pooling or average pooling. Pooling layers may be applied to each feature map separately. Pooling layers will typically be used between convolutional layers or following a convolutional layer.

A fully connected layer may include one or more fully connected layers. Nodes of the fully connected layer may fully connect to the nodes of the adjacent convolutional or pooling layer. An output layer may include one or more normalization and/or classification layers that may include classifier layers connected to a fully connected layer. In various embodiments, normalization and/or classification layers may include one or more of a SoftMax layer, SVM layer, regression layer, binary classifier layer, output/classier layer, or combination thereof.

In some embodiments, hidden layers may include one or more normalization layers, one or more nonlinear activation layers to increase nonlinearity, such as a ReLU layer, or both. Layers to increase nonlinearity include activation functions and will be associated with layers such as convolutional layers or fully connected layers. Thus, these layers identified herein may include such layers that apply activation functions such as ReLU, TanH, or sigmoid. In various embodiments, nodes of the input layer may be locally connected to a subset of nodes in an adjacent convolutional layer or pooling layer. In some embodiments, one or more fully connected layers may be included between the last pooling or convolutional layer and a classification layer wherein the nodes of the fully connected layer fully connect to the nodes of the adjacent pooling or convolutional layer. In one embodiment, a last fully connected layer fully connects to the one or more nodes of the output layer and the CNN does not include a classification layer. In various embodiments, a last classification layer may output probability scores, or a set of scores, for classes such as conditions, risks, or treatments. In some embodiments, an output layer includes a classifier that applies an activation function such as regression/logical regression, linear, SVM, or SoftMax. A SoftMax layer may provide probabilities for each prediction class for presentation to a further classification layer for output of a prediction. A SoftMax layer may provide normalization operations. In some embodiments, predictions having a threshold probability may be output or probabilities for all or a subset of the possible classes may be output. In some embodiments, an output layer includes a SoftMax layer and a classifier layer such as a binary classifier.

The AI model (e.g., AI model 608 of FIG. 6) or output layer thereof may provide predictions utilizing class probability values or scores. For example, a threshold score may indicate a maximum or minimum cut-off with respect to a prediction. In some embodiments, the output layer includes classification or classifier layers for classification of output values or scores with respect to the predictive classes. In some embodiments, the output layer of the AI model (e.g., AI model 608 of FIG. 6) may include a binary classifier that is executed to detect which of one or more classes, such as diseases or conditions, to include in the prediction or diagnosis. The AI model (e.g., AI model 608 of FIG. 6) may output a prediction of a disease or condition, an ancillary prediction with respect to a disease or condition, or another prediction, for example. In one embodiment, the AI model (e.g., AI model 608 of FIG. 6) may include an output layer comprising a SoftMax layer, which in some examples may be referred to as a normalization layer or classification layer. In a further embodiment, an output layer may include a SoftMax layer followed by a classification layer comprising a classifier. A classifier may detect or identify class labels or scores, for example. In one example, the classification layer may include a binary classifier. In one embodiment, the output layer includes a regression layer. A regression layer may be used for continuous output values. A regression layer may follow the last fully connected layer, for example. In one application, an output layer may include more than one separate output layer blocks connected to a single convolutional/pooling layer block.

In some embodiments, the system (e.g., system 600 or system 650 of FIG. 6) includes or utilizes an AI model (e.g., AI model 608) including one or more AI models comprising one or more neural networks (e.g., neural network 654), wherein one or more of the neural networks is a CNN, for the classification of corneal diseases. The neural network may include one or more convolutional layers, pooling layers, fully connected layers, or combinations thereof, for example. Convolutional layers may include various depths. Such layers may be stacked to form at least a portion of the network architecture of the neural network. Various architectures may be used. For example, various arrangements of convolutional and pooling layers block may be stacked. Additional layers such as rectifying layers, fully connected layers, an output layer comprising one or more normalization and/or classification layers, or combinations thereof may be used. In some examples, the AI model (e.g., AI model 608) includes multiple CNNs or portions thereof staked, e.g., vertically or horizontally, or may branch, converge, or diverge. Multiple neural networks may merge or share outputs wherein one or more of the neural networks is a CNN. In one example, an output of a first CNN may be provided as an input or may modify a parameter or weight of a second neural network or vice versa. Some embodiments of the system (e.g., system 600 or system 650) may analyze outputs of two neural networks to generate a prediction or output that considers at least a portion of the outputs of the first and second neural networks. In some embodiments, the AI model may include classical methods, which may be in addition to deep learning. For example, the AI model may include one or more deep learning submodels and one or more submodels utilizing classical AI methods or classifications.

As introduced above, the AI model may be trained to generate a prediction of diagnosis. In various embodiments, the prediction of diagnosis may include a set of scores. The model output may be a set of scores where each score is generated by a corresponding node in the output layer. The set of scores may include classification or category scores corresponding to particular category probabilities, such as condition, which may correspond to probability of the presence of a discrete category or a continuous scale value score, for example. The set of scores may be referred to as condition scores. The condition scores may correspond to an outcome related to one or more particular conditions or diseases of the cornea. For example, the AI model may associate each output node with a score which can be normalized using a SoftMax layer to give the likelihood of each category. In one embodiment, the set of scores is related to some medical condition. In these or other embodiments, each score may correspond to one or more of a prediction of severity of a medical condition or condition progression. In one example, the set of scores includes a set of condition scores wherein at least one of the scores represents a likelihood of a medical condition related to a corneal or anterior segment condition or disease.

An example training process set up according to various embodiments may include obtaining labeled data or annotating data to obtain labeled data. The labeled data may include training data such as annotated/labeled images or OCT maps. The method may include dividing the labeled data into subgroups where each has the same label that will be predicted by the neural network. Each data member of the group will have the same label (or set of annotations) and each label group represents a prediction category that will be predicted by the neural network. The number of data examples in each group is preferably similar so that the data is balanced to prevent bias in the training process toward a specific label. The training data set may include multiple frames for OCT images/maps. Optionally, all frames for each OCT image/map can be used. Although the frames represent the same cut, they may have different noise pattern and slightly different deformation of the cut. Thus, training with multiple frames may be used to make the neural network robust to noise. The number of layers and their parameters may be determined on basis of experiments and cross validation of the training data. Cross validation is done by dividing the training data into a training part and a validation part. The trained neural network is then used to test the validation part. This process may be repeated by dividing the training data into another different training set and another different validation set which is evaluated with the new trained network. Therefore, the whole training process may be repeated many times with different subsets of the training data and the trained network may be evaluated with different validation subsets. This may be done to ensure the robustness of the trained network. The trained network may then be evaluated using the real testing data.

Parameters of the model layers may be determined based on various criteria, e.g., input data or image size and/or results of the training experiments. The number of layers may be determined based on data size, e.g., number of images, results of the training experiments, and/or other criteria. In various embodiments, cross-validation employs exhaustive or non-exhaustive cross-validation such as hold-out, K-fold, or Monte Carlo cross-validation. For example, FIG. 10 shows a table indicating results of a 5-fold cross-validation in accordance with various embodiments of the present disclosure. The table indicates average accuracy by group for each of the normal cornea (98.1±3.9%), cornea with Fuch's dystrophy (93.7±10.8%), and cornea with keratoconus (99.0±1.9%).

### EXAMPLES

The following examples are illustrative of the disclosed methods. In light of this disclosure, those skilled in the art will recognize that variations of these examples and other examples of the disclosed method would be possible without undue experimentation.

### Example 1

Purpose: To design a convolutional neural network (CNN) for keratoconus detection using optical coherence tomography (OCT) corneal topography and thickness maps. (Combination of OCT corneal topography and thickness maps helps diagnose keratoconus using a convolutional neural network.)

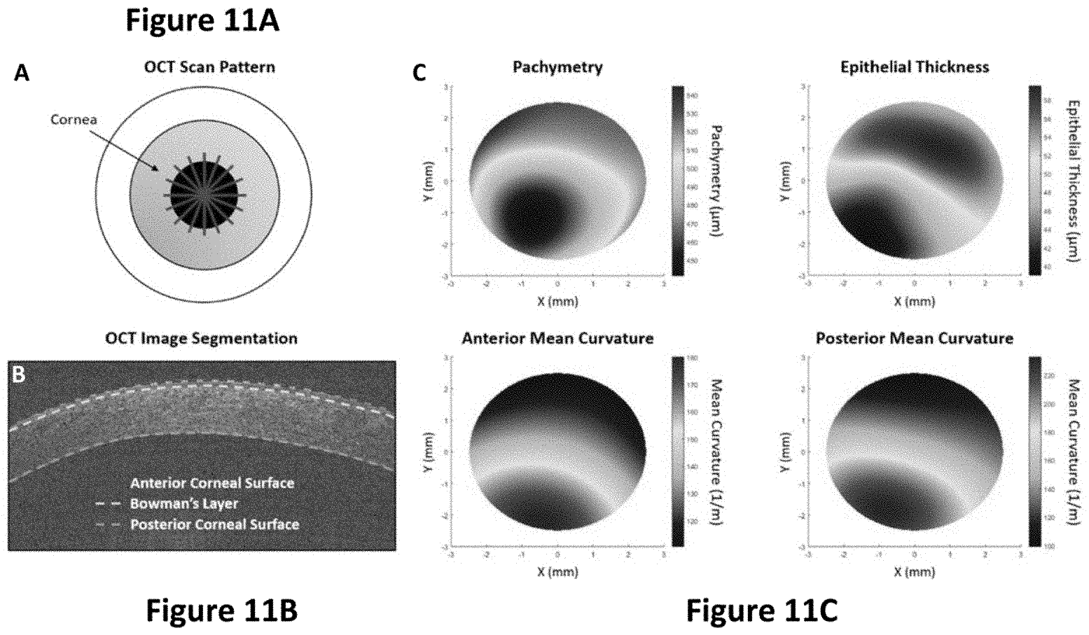

Subjects and methods: Normal subjects (n=52) and patients with either keratoconus (n=131) or contact lens-related warpage (n=20) were recruited. Keratoconus eyes were divided into 3 groups: 1) Manifest (n=89): slit-lamp or topographic signs of keratoconus and corrected distance visual acuity (CDVA)<20/20, 2) Subclinical (n=16): topographic signs of keratoconus but CDVA≥20/20, and 3) Forme fruste (n=26): normal-appearing eye with keratoconus in the contralateral eye. The central 6 mm of the cornea was imaged using a radial OCT scan pattern (Avanti, Optovue Inc.), and maps of pachymetry, epithelial thickness, anterior surface mean curvature, and posterior surface mean curvature were generated (Li et al, Ophthalmology, 2012; Pavlatos et al, BOE, 2020; FIG. 11). All maps were downsampled to a size of 16×16 pixels. The 4 map types were each treated as different color channels, and a grid search was used to optimize the network architecture and hyperparameters. Binary classification was performed to separate the keratoconus cases and non-keratoconus cases (normal or contact lens warpage). Repeated 5-fold cross-validation was used to evaluate model performance, and class activation maps were generated.

Results: The average balanced accuracy of the CNN during cross-validation was 94±2%. The precision and recall were 98±3% and 91±4%, respectively. The area under the receiver operating characteristic curve was 0.94±0.02. The network was able to detect 100% of the manifest and subclinical keratoconus cases. The accuracy for the forme fruste keratoconus cases was 56±19%. The network demonstrated good specificity, with 97±6% of normal eyes and 96±9% of warpage eyes being classified as non-keratoconus cases. The class activation maps indicated that the regions of the topography and thickness maps which contained the keratoconic cone were most important to the CNN for disease detection (FIG. 12).

Conclusions: OCT mapping of the cornea and CNNs can be used to detect keratoconus with high accuracy. This approach could be expanded to automate the classification of corneal diseases.

### Example 2

The AI based systems and methods described in the present disclosure may be implemented in an integrated system that is fully automated or assembled from different components that may require some manual intervention. In general, a system according to the present disclosure may comprise the components of a corneal topography measuring device capable of measuring and generating a corneal topography and an optical coherence tomography device, wherein both devices are capable of producing data in digital format or in a format that can be digitized, and a processing unit. The corneal topography measuring device may include, but not be limited to, Placido-ring topography, slit-scan corneal topography, Shiempflug-camera corneal tomography, raster photogrammetry, optical coherence tomography, or any other suitable cornea measuring devices known in the art. The processing unit may be a personal computer, a workstation, an embedded processor, or any other suitable data processing device commonly known in the art.

In addition to being implemented in a system, the AI based methods and systems of the present disclosure may also be provided in the form of software encoded on a computer readable medium for distribution to end users. Example computer media may include, but not be limited to, floppy disks, CD-roms, DVDs, hard drive disks, flash memory cards, downloadable files on an internet accessible server, or any other computer readable media commonly known in the art.

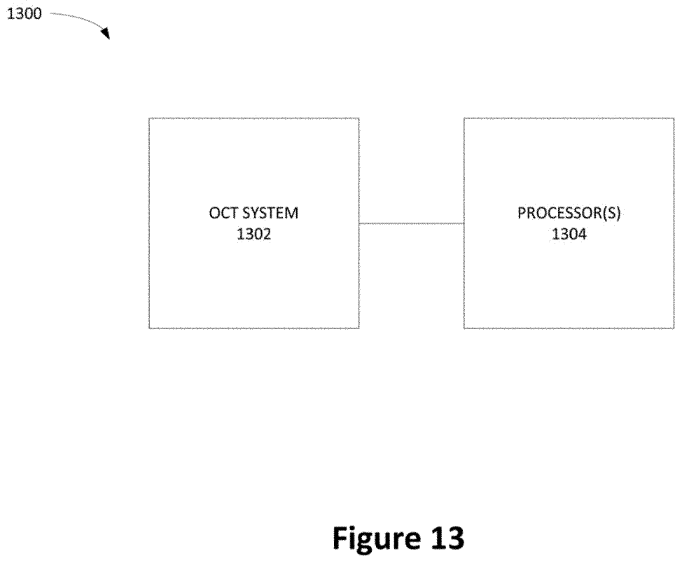

FIG. 13 schematically shows an example system 1300 for OCT image processing in accordance with various embodiments. System 1300 comprises an OCT system 1302 configured to acquire an OCT image comprising OCT interferograms and one or more processors or computing systems 1304 that are configured to implement the various processing routines described herein. OCT system 1300 can comprise an OCT system suitable for OCT angiography applications, e.g., a swept source OCT system or spectral domain OCT system.

In various embodiments, an OCT system can be adapted to allow an operator to perform various tasks. For example, an OCT system can be adapted to allow an operator to configure and/or launch various ones of the herein described methods. In some embodiments, an OCT system can be adapted to generate, or cause to be generated, reports of various information including, for example, reports of the results of scans run on a sample.

In embodiments of OCT systems comprising a display device, data and/or other information can be displayed for an operator. In embodiments, a display device can be adapted to receive an input (e.g., by a touch screen, actuation of an icon, manipulation of an input device such as a joystick or knob, etc.) and the input can, in some cases, be communicated (actively and/or passively) to one or more processors. In various embodiments, data and/or information can be displayed, and an operator can input information in response thereto.

In some embodiments, the above described AI based methods and systems can be tied to a computing system, including one or more computers. In particular, the methods and systems described herein, e.g., the systems depicted in FIG. 6 described above, can be implemented as a computer application, computer service, computer API, computer library, and/or other computer program product.

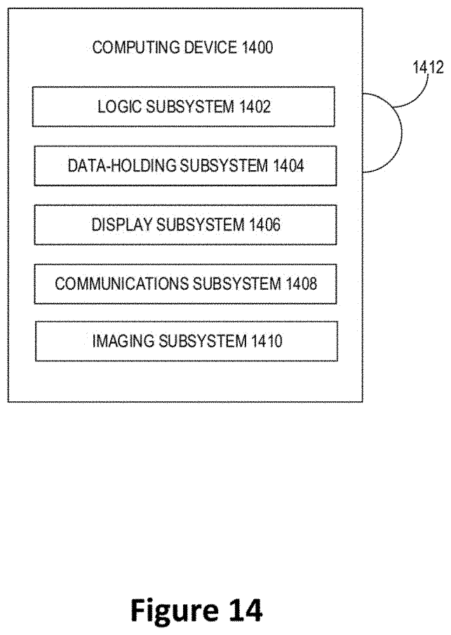

FIG. 14 schematically shows a non-limiting computing device 1400 that can perform one or more of the methods and processes described herein. For example, computing device 1400 can represent the processor 1304 included in system 1300 described above, and can be operatively coupled to, in communication with, or included in an OCT system or OCT image acquisition apparatus. Computing device 1400 is shown in simplified form. It is to be understood that virtually any computer architecture can be used without departing from the scope of this disclosure. In different embodiments, computing device 1400 can take the form of a microcomputer, an integrated computer circuit, printed circuit board (PCB), microchip, a mainframe computer, server computer, desktop computer, laptop computer, tablet computer, home entertainment computer, network computing device, mobile computing device, mobile communication device, gaming device, etc.

Computing device 1400 includes a logic subsystem 1402 and a data-holding subsystem 1404. Computing device 1400 can optionally include a display subsystem 1406, a communication subsystem 1408, an imaging subsystem 1410, and/or other components not shown in FIG. 14. Computing device 1400 can also optionally include user input devices such as manually actuated buttons, switches, keyboards, mice, game controllers, cameras, microphones, and/or touch screens, for example.

Logic subsystem 1402 can include one or more physical devices configured to execute one or more machine-readable instructions. For example, the logic subsystem can be configured to execute one or more instructions that are part of one or more applications, services, programs, routines, libraries, objects, components, data structures, or other logical constructs. Such instructions can be implemented to perform a task, implement a data type, transform the state of one or more devices, or otherwise arrive at a desired result.

The logic subsystem can include one or more processors that are configured to execute software instructions. For example, the one or more processors can comprise physical circuitry programmed to perform various acts described herein. Additionally or alternatively, the logic subsystem can include one or more hardware or firmware logic machines configured to execute hardware or firmware instructions. Processors of the logic subsystem can be single core or multicore, and the programs executed thereon can be configured for parallel or distributed processing. The logic subsystem can optionally include individual components that are distributed throughout two or more devices, which can be remotely located and/or configured for coordinated processing. One or more aspects of the logic subsystem can be virtualized and executed by remotely accessible networked computing devices configured in a cloud computing configuration.

Data-holding subsystem 1404 can include one or more physical, non-transitory, devices configured to hold data and/or instructions executable by the logic subsystem to implement the herein described AI based methods and processes. When such AI based methods and processes are implemented, the state of data-holding subsystem 1404 can be transformed (e.g., to hold different data).

Data-holding subsystem 1404 can include removable media and/or built-in devices. Data-holding subsystem 1404 can include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 1404 can include devices with one or more of the following characteristics: volatile, nonvolatile, dynamic, static, read/write, read-only, random access, sequential access, location addressable, file addressable, and content addressable. In some embodiments, logic subsystem 1402 and data-holding subsystem 1404 can be integrated into one or more common devices, such as an application specific integrated circuit or a system on a chip.

FIG. 14 also shows an aspect of the data-holding subsystem in the form of removable computer-readable storage media 1412, which can be used to store and/or transfer data and/or instructions executable to implement the herein described methods and processes. Removable computer-readable storage media 1412 can take the form of CDs, DVDs, HD-DVDs, Blu-Ray Discs, EEPROMs, flash memory cards, USB storage devices, and/or floppy disks, among others.

When included, display subsystem 1406 can be used to present a visual representation of data held by data-holding subsystem 1404. As the herein described AI based methods and processes change the data held by the data-holding subsystem, and thus transform the state of the data-holding subsystem, the state of display subsystem 1406 can likewise be transformed to visually represent changes in the underlying data. Display subsystem 1406 can include one or more display devices utilizing virtually any type of technology. Such display devices can be combined with logic subsystem 1402 and/or data-holding subsystem 1404 in a shared enclosure, or such display devices can be peripheral display devices.

When included, communication subsystem 1408 can be configured to communicatively couple computing device 1400 with one or more other computing devices. Communication subsystem 1408 can include wired and/or wireless communication devices compatible with one or more different communication protocols. As non-limiting examples, the communication subsystem can be configured for communication via a wireless telephone network, a wireless local area network, a wired local area network, a wireless wide area network, a wired wide area network, etc. In some embodiments, the communication subsystem can allow computing device 1400 to send and/or receive messages to and/or from other devices via a network such as the Internet.

When included, imaging subsystem 1410 can be used to acquire and/or process any suitable image data from various sensors or imaging devices in communication with computing device 1400. For example, imaging subsystem 1410 can be configured to acquire OCT image data, e.g., interferograms, as part of an OCT system, e.g., OCT system 1302 described above. Imaging subsystem 1410 can be combined with logic subsystem 1402 and/or data-holding subsystem 1404 in a shared enclosure, or such imaging subsystems can comprise periphery imaging devices. Data received from the imaging subsystem 1410 can be held by data-holding subsystem 1404 and/or removable computer-readable storage media 1412, for example.

It is to be understood that the configurations and/or approaches described herein are exemplary in nature, and that these specific embodiments or examples are not to be considered in a limiting sense, because numerous variations are possible. The specific routines or methods described herein can represent one or more of any number of processing strategies. As such, various acts illustrated can be performed in the sequence illustrated, in other sequences, in parallel, or in some cases omitted. Likewise, the order of the above-described processes can be changed.

The subject matter of the present disclosure includes all novel and nonobvious combinations and sub-combinations of the various processes, systems and configurations, and other features, functions, acts, and/or properties disclosed herein, as well as any and all equivalents thereof.

What is claimed is:

1. A computer-based method of classifying corneal shape abnormalities, the method comprising:
generating at least two corneal maps of a subject's cornea or a sublayer, wherein the at least two corneal maps include at least two of: a corneal topography map, a corneal thickness map, and a corneal reflectance map;
providing the at least two corneal maps as inputs to an artificial intelligence model comprising a neural network;
obtaining, via the neural network, one or more outputs related to presence of at least one condition, wherein the one or more outputs are generated based on the at least two corneal maps; and
classifying the subject's cornea based on the one or more outputs.

2. The method of claim 1, wherein the at least two corneal maps are generated based on an optical coherence tomography dataset.

3. The method of claim 1, wherein the neural network includes a convolutional neural network.

4. The method of claim 1, wherein the corneal topography map is a mean curvature map of an anterior surface, a posterior surface, or a sub-epithelial surface of the cornea.

5. The method of claim 1, wherein the corneal topography map is an elevation map of an anterior surface, a posterior surface, or a sub-epithelial surface of the cornea.

6. The method of claim 1, wherein the corneal topography map is an axial power map of an anterior surface, a posterior surface, or a sub-epithelial surface of the cornea.

7. The method of claim 1, wherein the corneal topography map is a tangential power map of an anterior surface, a posterior surface, or a sub-epithelial surface of the cornea.

8. The method of claim 1, wherein the corneal thickness map included in the inputs includes one or more of an overall corneal thickness map, a corneal epithelial thickness map, a corneal stromal thickness map, a Descemet's layers thickness map, or a corneal endothelial thickness map.

9. The method of claim 1, wherein the inputs indicate reflectance variation at different depths of corneal thickness, and wherein the one or more outputs include an indication of one or more depth-dependent irregularities in reflectance intensity.

10. The method of claim 1, wherein the subject's cornea is classified as normal, keratoconic, or cornea with Fuch's dystrophy based on the one or more outputs.

11. A system for classifying corneal shape abnormalities, the system comprising:
an interface to a neural network; and
one or more processors executing computer program instructions that, when executed, cause the one or more processors to:
generate a plurality of maps of a subject's cornea;
provide inputs to the neural network via the interface, wherein the inputs include the maps or metrics derived from the maps;
obtain, from the neural network via the interface, one or more outputs related to presence of at least one condition, wherein the neural network is to generate the one or more outputs based on the maps and/or the metrics; and
classify the subject's cornea based on the one or more outputs generated by the neural network.

12. The system of claim 11, wherein one or more of the maps are generated based on an optical coherence tomography (OCT) dataset.

13. The system of claim 12, wherein the maps include one or more maps of corneal shape and one or more maps of corneal thickness.

14. The system of claim 12, wherein the maps include one or more maps of corneal shape and one or more maps of corneal reflectance.

15. The system of claim 12, wherein the maps include one or more maps of corneal thickness and one or more maps of corneal reflectance.

16. The system of claim 12, wherein the one or more outputs include an indication of one or more depth-dependent irregularities in reflectance intensity based on the OCT dataset.

17. The system of claim 11, wherein the plurality of maps include one or more of a mean curvature map, an elevation map, a float map, an axial power map, or a tangential power map of an anterior surface, a posterior surface, or a sub-epithelial surface of the cornea.

18. The system of claim 11, wherein the plurality of maps include one or more of an overall corneal thickness map, a corneal epithelial thickness map, a corneal stromal thickness map, a Descemet's layers thickness map, or a corneal endothelial thickness map of the cornea.

19. The system of claim 11, wherein the plurality of maps include one or more of a corneal epithelial reflectance map, a Bowman's layer reflectance map, a corneal stromal reflectance map, a Descemet's layer and endothelial reflectance map, or a guttae reflectance maps of the cornea.

20. The system of claim 11, wherein the maps include maps obtained using different imaging modalities.

21. The system of claim 11, wherein the metrics include a plot of reflectance variation at different depths of corneal thickness.

22. The system of claim 11, wherein the subject's cornea is classified as normal, keratoconic, or cornea with Fuch's dystrophy based on the one or more outputs generated by the neural network.
